(19) United States
(12) Patent Application Publication
(10) Pub. No.: US 2014/0188399 A1
(43) Pub. Date: Jul. 3, 2014

(54) ELECTRONIC ALCOHOL INTOXICATION DETECTION, IDENTIFICATION AND SUBSCRIBER NOTIFICATION SYSTEM

(71) Applicant: Carlos Alberto Valenzuela, Rockville, MD (US)

(72) Inventor: Carlos Alberto Valenzuela, Rockville, MD (US)

(21) Appl. No.: 14/142,728

(22) Filed: Dec. 27, 2013

Related U.S. Application Data
(60) Provisional application No. 61/746,447, filed on Dec. 27, 2012.

(51) Int. Cl. G01N 33/497; G08C 17/02

(52) U.S. Cl. CPC G01N 33/4972; G08C 17/02
USPC 702/19

(57) ABSTRACT

An electronic alcohol intoxication detection, identification and subscriber notification system may include a device that may include an enclosure having an interior housing and an exterior. The enclosure houses components indentifying intoxication level data. A remote database may wirelessly connect to the intoxication level data. A virtual server may than transmit the intoxication level data in the form of notifications to at least one contact point of a subject.

### CROSS-REFERENCE TO RELATED APPLICATION

This application claims the benefit of U.S. provisional application No. 61/746,447, filed Dec. 27, 2012, the contents of which are herein incorporated by reference.

### BACKGROUND OF THE INVENTION

The present invention relates to alcohol intoxication detection and, more particularly, to an electronic alcohol intoxication detection, identification and subscriber notification system.

Every year, millions of people are involved in alcohol related accidents/incidents due to driving and/or working while under the influence of alcohol. Currently, devices or systems that are used administer the test in the field, which means a potentially drunk driver is already on the road and endangering themselves and others. In the workplace, intoxication is most often undetected and there is no system in place to monitor and notify the proper people. Current devices and systems only notify of blood alcohol level and do not deter the action of the potential drunk driver/employee.

As can be seen, there is a need for an electronic alcohol intoxication detection, identification and subscriber notification system that allows for testing of subjects in proactive steps, notifying proper people, providing transportation as well as assessing the safety of workers on the job.

### SUMMARY OF THE INVENTION

In one aspect of the present invention, an electronic alcohol intoxication detection, identification and subscriber notification system comprises: an enclosure having an interior housing and an exterior, wherein the enclosure houses components indentifying intoxication level data; a remote database wirelessly connected to the intoxication level data; and a virtual server that transmits the intoxication level data in the form of notifications to at least one contact point of a subject.

In another aspect of the present invention, a electronic alcohol intoxication detection, identification and subscriber notification device comprises: an enclosure having an interior housing and an exterior, wherein the exterior houses a display screen monitor and an intoxication identification device, wherein the interior housing comprises: an electronic bread board; a micro motherboard shield attached to the electronic bread board, wherein the micro motherboard shield has an internal power and Ethernet capability; a central processing unit motherboard attached to the electronic bread board; a random access memory chip attached to the electronic bread board; a wireless carrier card attached to the electronic bread board; a hard drive attached to the electronic bread board; a compact flash drive attached to the electronic bread board; an antenna attached to the electronic bread board; a radio frequency identification scanner attached to the electronic bread board; a Bluetooth transceiver attached to the electronic bread board; an alcohol sensor kit attached to the electronic bread board; and a series of light emitting diodes.

In another aspect of the present invention, a method of notifying a subscriber of an intoxicated subject comprises: providing a subject with a device comprising an enclosure having an interior housing and an exterior, wherein the exterior houses a display screen monitor and an intoxication identification device, wherein the interior housing comprises: an electronic bread board; a micro motherboard shield attached to the electronic bread board, wherein the micro motherboard shield has an internal power and Ethernet capability; a central processing unit motherboard attached to the electronic bread board; a random access memory chip attached to the electronic bread board; a wireless carrier card attached to the electronic bread board; a hard drive attached to the electronic bread board; a compact flash drive attached to the electronic bread board; an antenna attached to the electronic bread board; a radio frequency identification scanner attached to the electronic bread board; a Bluetooth transceiver attached to the electronic bread board; an alcohol sensor kit attached to the electronic bread board; and a series of light emitting diodes; collecting personal data and subscriber contact information from the subject; processing an input sample data from the subject; displaying an intoxication level data of the subject on the display screen monitor; delivering intoxication level data to a remote database; and sending notification of intoxication level data to a subscriber.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention;

FIG. 2 is a flow chart of an exemplary embodiment of the present invention; and

FIG. 3 is a schematic view of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides an electronic alcohol intoxication detection, identification and subscriber notification system that may include a device that may include an enclosure having an interior housing and an exterior. The enclosure houses components indentifying intoxication level data. A remote database may wirelessly connect to the intoxication level data. A virtual server may than transmit the intoxication level data in the form of notifications to at least one contact point of a subject.

The present invention may be an electronic device that may be a layer of awareness between an alcohol serving establishment and other applicable persons, organizations, employers and a potentially drunk patron/employee. The present invention may deter the potentially drunk patron/employee from driving or working while under the influence of alcohol. Data may be produced that may help with studies on alcohol consumption in general, and with the subject. The system may also provide at least one contact point of a subject with information on the subject.

The present invention may include at least one computer with a user interface. The computer may include any computer including, but not limited to, a desktop, laptop, and smart device, such as, a tablet and smart phone. The computer includes a program product including a machine-readable program code for causing, when executed, the computer to perform steps. The program product may include software which may either be loaded onto the computer or accessed by the computer. The loaded software may include an application on a smart device. The software may be accessed by the computer using a web browser. The computer may access the software via the web browser using the internet, extranet, intranet, host server, internet cloud and the like.

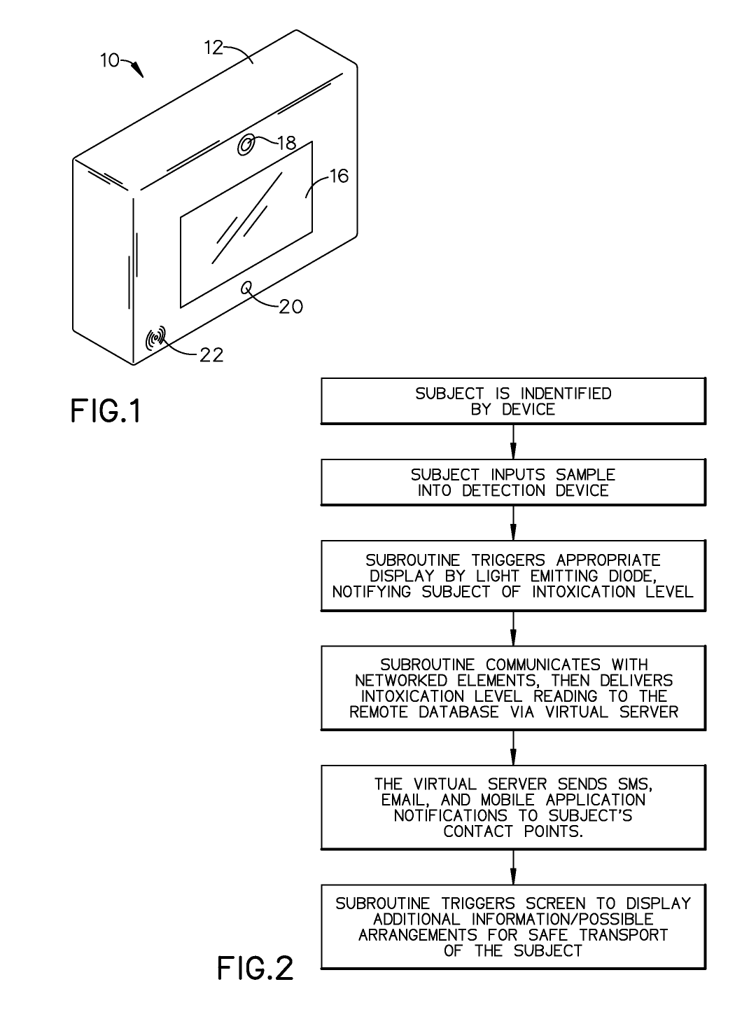

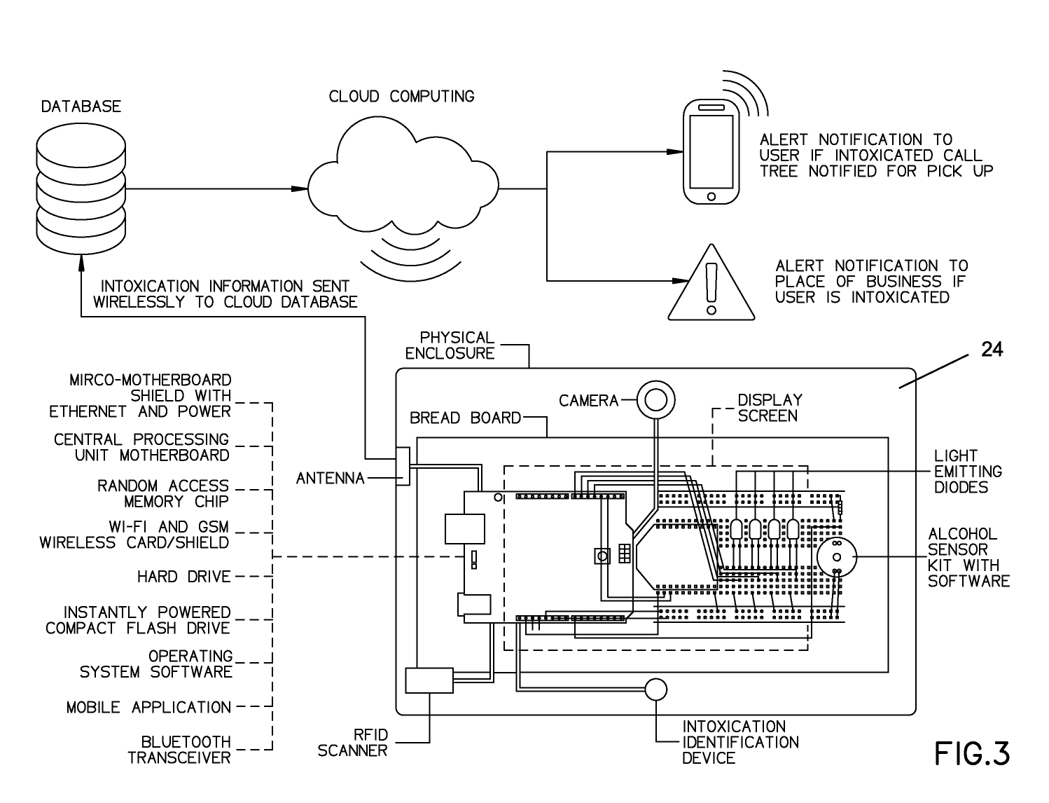

As is illustrated in FIGS. 1 through 3, the electronic alcohol intoxication detection, identification and subscriber notification system may include a device 10 that may include an enclosure 12. In certain embodiments the device 10 may be wireless. In certain embodiments, the enclosure 12 may be a box with dimensions of approximately 18 inches×12 inches×5 inches. The enclosure 12 may be made from a plastic or the like. The enclosure 12 may hold assembled components within an interior housing 24. The interior housing 24 of the enclosure 12 may include, but not limited to, a micro motherboard shield with Ethernet and power, a central processing unit motherboard, a random access memory chip, a wireless carrier card, a hard drive, a compact flash drive, a radio frequency identification (RFID) 175 mhz scanner 22, an intoxication identification device 20, an alcohol sensor kit with software, communication availability for a mobile application, light emitting diode (LED) lights, an antenna, and a Bluetooth transceiver and an electronic bread board as a base.

In certain embodiments, a micro motherboard shield with built in Ethernet and power may enable the device 10 to transfer processed data to a virtual server. A central processing unit (CPU) motherboard may be able to convert data into readable and displayable data for distribution and notification. In certain embodiments, a random access memory (RAM) chip may be used. In certain embodiments, the RAM chip may store approximately 128 MB of data. The Wi-Fi and global system for mobile communications (GSM)/wireless carrier card/shield may allow connectivity for the micro motherboard shield to transmit data to the virtual server. An antenna may allow for better Wi-Fi connection, reception, and transmission of the data. In certain embodiments a display screen monitor 16 may be attached of the enclosure 12 and connected to the micro motherboard shield. In certain embodiments, the display screen monitor 16 may be an approximately 5 inch×5 inch TFT LCD (Touch Screen) color video monitor 16 and graphics video drivers may allow the screen to be read for following sampling instructions and testing results. A hard drive may provide program memory space on the CPU motherboard. An instantly powered compact flash drive may be in the interior housing 24 of the enclosure 12 for photography storage space. A Bluetooth transceiver may be available to detect non subscribers via a mobile device. Operating system software may be a set of if/then and subroutine coding in order to make the entire system function as listed. An alcohol sensor kit with an application programming interface may be provided to gather sample data for processing to let the subject become aware of intoxication above the legal limit. A radio frequency identification (RFID) 175 mhz scanner 22 may be connected for the subject to scan personal data and contact information into the system for further uploading to appropriate subscriber end points which become the at least one contact point of the subject. The software may include a secondary set of commands and routines to allow components of the enclosure 12 to work properly together for the purpose intended. A micro camera 18 may work as a subject identification system for test correspondence. An electronic bread board may be the base for the attachment of all identified elements that function together within the interior housing 24 of the enclosure 12. A series of light emitting diode (LED) lights may be included and activated if the subject may be intoxicated above a legal limit after a sample input. A subroutine may trigger appropriate display of the LEDs, which then may notify the subject of intoxication. The subroutine may then communicate with networked elements such as Ethernet, Wi-Fi, GSM and the like. The subroutine may then deliver intoxication level readings to a remote database via the virtual server. The virtual server may then send short message service (sms) messages, email, mobile phone app notification or the like to at least one contact point of the subject. Then the subroutine may trigger the screen to display additional information/ possible arrangements for safe transport of the subject from the establishment or workplace.

In certain embodiments, the enclosure 12 may have various shapes and sizes as long as the enclosure may hold all the components. In alternate embodiments the micro camera 18 may be included and the lens may extend out from an exterior of the enclosure 12. The use of the micro camera 18 within the system may also allow for the identification of subjects as they enter a work environment. This identification may be in order to ensure the safety of the employee and the other employees.

In certain embodiments, the remote database and virtual server may be responsible for the transmission of data to subscriber end points. A mobile application may be available for download to better enable the subject to track results of the subject tests and enable support.

A method of using the electronic alcohol intoxication detection, identification and subscriber notification system may include the following. The system may be used in a location such as a nightclub, restaurant, prom party, wedding, places of employment, and the like. These places are particularly prone to the position of people being under the influence of alcohol and may create dangerous and/or life threatening situations by drinking and driving. In certain embodiments, the system may be used at alcohol and counseling rehab centers for identification of a relapsed subject. The enclosure 12 may be placed in a particular location. The subject may be identified by the device 10. The subject may use the alcohol sensor kit that may identify the alcohol level within the subject. The subject may input a sample into the device 10 so that the alcohol sensor kit may function. A subroutine may trigger appropriate display on the display screen monitor 16 by LEDs which may indicate whether the value is under or over a legal limit. The display screen monitor 16 may also indicate the intoxication level of the subject. The subroutine may communicate with networked elements. The networked elements may be through Bluetooth, a mobile application, a Wi-Fi connection to a computer such as a mobile smart phone and the like. The subroutine may then deliver the data to the remote database via a virtual server. The database may allow access to the data through Bluetooth, a mobile application, Wi-Fi connection to a computer such as a mobile smart phone and the like. A notification may be sent by SMS, email, mobile application notifications and the like to the at least one contact point of the subject. An alert may be sent to the place of business as well to indicate the alcohol level of the subject. The subroutine may also trigger the display screen monitor 16 to display additional information and possible arrangements for safe transport of the subject, such as local taxi service information and the like.

The computer-based data processing system and method described above is for purposes of example only, and may be implemented in any type of computer system or programming or processing environment, or in a computer program, alone or in conjunction with hardware. The present invention may also be implemented in software stored on a computer-readable medium and executed as a computer program on a general purpose or special purpose computer. For clarity, only those aspects of the system germane to the invention are described, and product details well known in the art are omitted. For the same reason, the computer hardware is not described in further detail. It should thus be understood that the invention is not limited to any specific computer language, program, or computer. It is further contemplated that the present invention may be run on a stand-alone computer system, or may be run from a server computer system that can be accessed by a plurality of client computer systems interconnected over an intranet network, or that is accessible to clients over the Internet. In addition, many embodiments of the present invention have application to a wide range of industries. To the extent the present application discloses a system, the method implemented by that system, as well as software stored on a computer-readable medium and executed as a computer program to perform the method on a general purpose or special purpose computer, are within the scope of the present invention. Further, to the extent the present application discloses a method, a system of apparatuses configured to implement the method are within the scope of the present invention.

It should be understood, of course, that the foregoing relates to exemplary embodiments of the invention and that modifications may be made without departing from the spirit and scope of the invention as set forth in the following claims.

What is claimed is:

1. An electronic alcohol intoxication detection, identification and subscriber notification system comprising:
an enclosure having an interior housing and an exterior, wherein the enclosure houses components indentifying intoxication level data;
a remote database wirelessly connected to the intoxication level data; and
a virtual server that transmits the intoxication level data in the form of notifications to at least one contact point of a subject.

2. The electronic alcohol intoxication detection, identification and subscriber notification system of claim 1, further comprising an alert to a place of business if the subject is intoxicated.

3. The electronic alcohol intoxication detection, identification and subscriber notification system of claim 1, wherein the virtual server transmits the intoxication level data wirelessly.

4. An electronic alcohol intoxication detection, identification and subscriber notification device comprising:
an enclosure having an interior housing and an exterior, wherein the exterior houses a display screen monitor and an intoxication identification device, wherein the interior housing comprises:
an electronic bread board;
a micro motherboard shield attached to the electronic bread board, wherein the micro motherboard shield has an internal power and Ethernet capability;
a central processing unit motherboard attached to the electronic bread board;
a random access memory chip attached to the electronic bread board;
a wireless carrier card attached to the electronic bread board;
a hard drive attached to the electronic bread board;
a compact flash drive attached to the electronic bread board;
an antenna attached to the electronic bread board;
a radio frequency identification scanner attached to the electronic bread board;
a Bluetooth transceiver attached to the electronic bread board;
an alcohol sensor kit attached to the electronic bread board; and
a series of light emitting diodes.

5. The electronic alcohol intoxication detection, identification and subscriber notification device of claim 4, further comprising a micro camera housed by the enclosure.

6. A method of notifying a subscriber of an intoxicated subject comprising:
providing a subject with a device comprising an enclosure having an interior housing and an exterior, wherein the exterior houses a display screen monitor and an intoxication identification device, wherein the interior housing comprises: an electronic bread board; a micro motherboard shield attached to the electronic bread board, wherein the micro motherboard shield has an internal power and Ethernet capability; a central processing unit motherboard attached to the electronic bread board; a random access memory chip attached to the electronic bread board; a wireless carrier card attached to the electronic bread board; a hard drive attached to the electronic bread board; a compact flash drive attached to the electronic bread board; an antenna attached to the electronic bread board; a radio frequency identification scanner attached to the electronic bread board; a Bluetooth transceiver attached to the electronic bread board; an alcohol sensor kit attached to the electronic bread board; and a series of light emitting diodes;
collecting personal data and subscriber contact information from the subject;
processing an input sample data from the subject;
displaying an intoxication level data of the subject on the display screen monitor;
delivering intoxication level data to a remote database; and
sending notification of intoxication level data to a subscriber.

7. The method of claim 6, further comprising displaying additional information on the display screen monitor for safe transport of the subject.
